(19) United States
(12) Patent Application Publication
(10) Pub. No.: US 2002/0165207 A1
(43) Pub. Date: Nov. 7, 2002

(54) Compositions and methods for the treatment of diabetic neuropathy

(76) Inventor: Richard Rosenbloom, Elkins Park, PA (US)

Correspondence Address: Knoble & Yoshida, LLC, Eight Penn Center, Suite 1350, 1628 John F. Kennedy Blvd., Philadelphia, PA, 19103, US

(21) Appl. No.: 09/847,121

(22) Filed: May 2, 2001

(51) Int. Cl. A61K 31/59; A61K 31/7042; A61K 31/353

(52) U.S. Cl. 514/168; 514/25; 514/27; 514/456

(57) ABSTRACT

Compositions and a method for the treatment of diabetic neuropathy is disclosed. The compositions comprise a mixture of a compound that promotes synthesis of nerve growth factor, an aldose reductase inhibitor and an antioxidant, optionally formulated in a pharmaceutically acceptable carrier. This combination of active agents provides significant, effective relief of the symptoms of diabetic neuropathy, as well as at least partial recovery of lost neurological function in some cases. In addition, the compositions of the present invention, when used in effective amounts to treat diabetic neuropathy, do not exhibit the severe side effects of many prior art compositions proposed for treatment of this ailment.

In a second aspect, a method for the administration of a composition in accordance with the present invention for the treatment of diabetic neuropathy is disclosed. In the method, an effective amount of the composition of the invention is administered on a regular basis over a period of time sufficient to provide the beneficial effects of relief from the symptoms of diabetic neuropathy, as well as at least some recovery of the damaged nerve tissues.

### BACKGROUND OF THE INVENTION

1. Field of the Invention

The present invention relates to compositions and methods for the treatment of diabetic neuropathy.

2. Description of the Prior Art

Diabetes mellitus is a common disease that is usually classified into insulin-dependent and non-insulin dependent types. Both types may be managed by diet, in combination with insulin in the first type and a variety of drugs in the second type. However, while the changes in blood glucose associated with diabetes can usually be managed reasonably satisfactorily by conscientious patients and doctors, this does not prevent long term damage to many tissues as a result of the disease. This damage may take many forms but the major types are damage to the eyes (retinopathy), nerves (neuropathy), kidneys (nephropathy) and cardiovascular system.

There are many approaches to reducing or preventing these forms of damage, which are collectively known as the long-term complications of diabetes. One approach is based on damage that results from over-production of the glucose metabolite, sorbitol, in the cells of the body. Glucose can be converted to sorbitol by the enzyme aldose reductase. High levels of sorbitol may be among the causes of diabetic complications such as diabetic neuropathy. As a result, a number of pharmaceutical companies have been developing aldose reductase inhibitors for the purpose of reducing diabetic neuropathy.

It has been established that a wide variety of flavanoids are effective inhibitors of aldose reductase, including such flavanoids as quercetin, quercetrin and myrecetrin. However, U.S. Pat. No. 4,232,040 discloses that despite the fact that these flavanoids have been shown in in vitro studies to be among the most potent flavanoids for aldose reductase inhibition, a need exists for aldose reductase inhibitors that can be more effectively used and in lower doses than the prior art compounds, including these flavanoids.

In fact, numerous patents are devoted to goal of developing improved aldose reductase inhibitors. Among these patents are U.S. Pat. Nos. 6,069,168; 5,011,840; 4,210,667; 4,147,795; 5,866,578; and 5,561,110. Numerous other patents exist which relate to aldose reductase inhibitors.

Another approach to the treatment of diabetic neuropathy is disclosed in U.S. Pat. No. 5,840,736 (Zelle et al.). In this method, pharmaceutical compositions are disclosed for stimulating the growth of neurites in nerve cells. The compositions include a neurotrophic amount of a compound and a nerve growth factor. These compositions may be administered in a number of ways including orally and topically.

Still another approach to the treatment of neuropathy is disclosed in U.S. Pat. No. 5,550,249 (Della Ville et al.). In this approach, compositions suitable for treatment of vitamin H deficiencies are administered for the treatment of neuropathy. This patent relates to biotin salts with alkanolamines. The compositions may be administered orally, parenterally or topically.

U.S. Pat. No. 5,665,360 (Mann) relates to the treatment of peripheral neuropathies associated with diabetes mellitus by periodic topical application of a composition containing capsicum oleoresin as the active ingredient. When applied to the skin of the affected area, pain and burning associated with the neuropathy are said to be reduced. However, capsicum oleoresin has been shown to kill nerve endings in some cases and thus this composition suffers from this disadvantage.

U.S. Pat. No. 5,981,594 (Okamoto et al.) relates to a method of treatment of diabetic neuropathy using combined administration of a formulation including as an active ingredient, a prostaglandin I derivative with an anti-diabetic agent in order to improve nerve conduction velocities. Suitable anti-diabetic agents include oral hypoglycemic agents and insulin.

The Okamoto patent also contains a detailed discussion of the various types of neuropathy that may be associated with diabetes. According to this patent, nerve conduction velocity (NCV) is the most widely used method of objectively evaluating the severity of diabetic neuropathy. This patent also mentions that current methods of treating diabetic neuropathy such as dietetic therapy, administration of insulin, administration of aldose reductase inhibitors or aminoguanidine to improve abnormal glucose metabolism, administration of troglitazone or agents for the improvement of blood flow have been tested but found to be insufficient when a single drug was used. Also, according to this patent, methods of treatment by combined use of different therapeutic agents which have different functions had yet to be established. The patent concludes that combined drug therapies for diabetic neuropathy, aiming at recovering once reduced nerve conduction velocity, have not yet been confirmed.

There remains a need in the art for an effective treatment for diabetic neuropathy that does not suffer from the disadvantage that it causes severe side effects, as do many aldose reductase inhibitors, for example.

Accordingly, it is the primary object of the present invention to provide oral or parenteral compositions that are effective for the treatment of diabetic neuropathy.

It is another object of the present invention to provide compositions for the treatment of diabetic neuropathy that do not cause severe side effects in the patients treated with the compositions.

These and other objects of the present invention will be apparent from the summary and detailed descriptions of the invention that follow.

### SUMMARY OF THE INVENTION

In a first aspect, the present invention relates to compositions for the treatment of diabetic neuropathy. The compositions comprise a mixture of a compound that promotes synthesis of nerve growth factor, an aldose reductase inhibitor and an antioxidant, optionally formulated in a pharmaceutically acceptable carrier. It has been found that this combination of active agents provides significant, effective relief of the symptoms of diabetic neuropathy, as well as at least partial recovery of lost neurological function in some cases. In view of the consensus in the art that effective combinations of various active agents have not been demonstrated to be effective for the treatment of diabetic neuropathy, the present invention provides a surprising and unexpected effect. In addition, the compositions of the present invention, when used in effective amounts to treat diabetic neuropathy, do not exhibit the severe side effects of many prior art compositions proposed for treatment of this ailment.

In a second aspect, the present invention relates to a method for the administration of a composition in accordance with the present invention for the treatment of diabetic neuropathy. In the method, an effective amount of the composition of the invention is ingested on a regular basis over a period of time sufficient to provide the beneficial effects of relief from the symptoms.

### DETAILED DESCRIPTION OF THE PREFERRED EMBODIMENTS

In a first aspect, the present invention relates to compositions for the treatment of diabetic neuropathy. The compositions include a compound that promotes synthesis of nerve growth factor, an aldose reductase inhibitor and an antioxidant, optionally formulated in a pharmaceutically acceptable carrier.

The compound that promotes synthesis of nerve growth factor may be selected from suitable compounds that have been shown to have this activity. Suitable compounds that promote synthesis of nerve growth factor are those that do not induce significant, adverse side effects when ingested by a patient in amounts that promote synthesis of nerve growth factor, and which do not react with one or more of the ingredients of the compositions resulting in a substantial loss of activity of one or more active ingredients. Preferred compounds for promoting synthesis of nerve growth factor are those that occur naturally in the human body and/or materials obtained from plants or animal or derivatives thereof, which may be ingested by humans without significant, adverse side effects in the amounts used.

Exemplary compounds that promote synthesis of nerve growth factor are vitamin D₃, vitamin D₃ derivatives such as 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene. The preferred nerve growth factor used in the compositions of the present invention is vitamin D₃. Also, pharmaceutically acceptable salts of the compounds that promote synthesis of nerve growth factor may be employed. As used in this specification, derivatives refers to compounds which possess at least one structural moiety in common with the compound from which they are derived, which common structure is a characterizing structural element of the compound from which the derivative is derived.

The compound that promotes synthesis of nerve growth factor is used in an amount effective to promote the synthesis of nerve growth factor of about 6-14.3 IU per kg of body weight of the patient. More preferably, the compound that promotes synthesis of nerve growth factor is employed in an amount of about 8-14.3 IU per kg body weight of the patient, and most preferably an amount of 10-13 IU is used per kg of body of the patient.

The preferred compounds that induce synthesis of nerve growth factor may, in addition to this activity, also function to prevent neurotrophic deficits. This additional effect of the preferred compounds may also contribute to the overall beneficial effect of the compositions of the present invention.

In order to formulate the compound that promotes synthesis of nerve growth factor in the compositions of the present invention, it may be necessary to use a dispersant. Suitable dispersant materials are known to persons skilled in the art. A particularly suitable dispersant for the compounds that promote synthesis of nerve growth factor is corn oil. Corn oil also has the advantage that it is a natural product. The amount of corn oil used is an amount sufficient to disperse the compound that promotes synthesis of nerve growth factor.

The second active ingredient of the compositions of the present invention is an aldose reductase inhibitor. Numerous suitable aldose reductase inhibitors are known to persons skilled in the art. Again, suitable aldose reductase inhibitors are those that do not induce significant, adverse side effects when administered to a patient in an amount effective for aldose reductase inhibition, and which do not react with one or more of the ingredients of the composition resulting in a substantial loss of activity of one or more active ingredients of the composition. Preferred aldose reductase inhibitors are those that occur naturally in the human body and/or materials obtained from plants or animal or derivatives thereof, which may be administered to humans without significant, adverse side effects in the amounts used.

As mentioned above, numerous aldose reductase inhibitors are known to persons skilled in the art. However, significant adverse side effects are associated with the use of many aldose reductase inhibitors in humans. Thus, it is important to select one or more aldose reductase inhibitors for use in the compositions of the present invention based on minimizing the risk associated with use of the aldose reductase inhibitor taking into account the amount of that particular inhibitor that must be employed to achieve the desired level of aldose reductase inhibition. Different aldose reductase inhibitors exhibit different levels of inhibition. With this in mind, the preferred aldose reductase inhibitors for use in the compositions of the present invention are flavonoids and flavonoid derivatives. Exemplary aldose reductase inhibitors include (−)-epigallocatechin; (−)-epigallocatechin-gallate; 1,2,3,6,-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; and wogonin.

The most preferred flavonoid and/or flavonoid derivative aldose reductase inhibitors are quercetin, quercetrin, myricetin, kaempferol and myrecetrin since these compounds exhibit a high level of aldose reductase inhibition in combination with a relatively low toxicity. Also, pharmaceutically acceptable salts of these aldose reductase inhibitors may be employed. The particular aldose reductase inhibitor included in the compositions may be determined by factors such as toxicity, bioavailability, solubility or dispersability, among others.

The flavonoids and flavonoid derivatives are also preferred since some of these compounds may provide additional beneficial effects in the composition of the present invention. For example, quercetin may act as a chelator for transition metals that some studies have linked to certain symptoms of diabetic neuropathy. Flavonoids may also have some anti-inflammatory activity and/or may help stabilize cell membranes, both of which activities may be beneficial in the treatment of diabetic neuropathy.

The aldose reductase inhibitor is used in an amount that provides substantially the same level of aldose reductase inhibition as 13-21.4 mg/kg body weight of the patient per day of quercetin. More preferably, the aldose reductase inhibitor is employed in an amount that provides substantially the same level of aldose reductase inhibition as 17.2-21.4 mg/kg body weight of the patient per day of quercetin and most preferably an amount that provides substantially the same level of aldose reductase inhibition as 18-21 mg/kg body weight of the patient per day of quercetin.

Another active ingredient in the compositions of the present invention is the antioxidant. The antioxidant may be a single compound or a mixture of two or more compounds. Also, the antioxidant may include one or more compounds that provide additional beneficial effects beyond the antioxidant activity, such as aldose reductase inhibition.

Compounds which may be used as antioxidants are those which exhibit antioxidant activity when ingested without causing any severe adverse side affects when used in an amount effective to provide sufficient antioxidant activity, and which do not react with one or more of the ingredients of the compositions resulting in a substantial loss of activity of one or more active ingredients. Preferred antioxidants are those that occur naturally in the human body and/or materials obtained from plants or animal or derivatives thereof which may be ingested or topically applied by humans without significant, adverse side effects in the amounts used.

More preferred antioxidants are selected from ascorbyl palmitate, ascorbic acid (vitamin C), vitamin A, vitamin E, α-lipoic acid, especially DL-α-lipoic acid, coenzyme Q10, glutathione, catechin, glangin, rutin, luteolin, morin, fisetin, silymerin, apigenin, gingkolides, hesperitin, cyanidin, citrin and derivatives thereof which exhibit antioxidant activity. Even more preferably, mixtures of two or more antioxidants are employed in the composition of the present invention. Particularly preferred antioxidant mixtures are ascorbyl palmitate with one or both of vitamin A and vitamin E as tocopherols or vitamin E as mixed tocopherols. Most preferably, all-natural vitamin E tocopherols are employed. The antioxidants may also be used in the form of their pharmaceutically acceptable salts and this may be preferred in some cases to increase solubility or dispersability, to reduce adverse side effects, to increase bioavailability, etc.

Ascorbyl palmitate may be used in amounts of 11-28.6 mg/kg body weight of the patient per day. More preferably, ascorbyl palimitate is used in amounts of 14.3-28.6 mg/kg body weight of the patient per day. Most preferably ascorbyl palmitate is used in amounts of 16-26 mg/kg body weight of the patient.

When vitamin E is employed as mixed tocopherols, an amount of about 4-11.4 IU per kg body weight of the patient, per day, may be employed. More preferably, about 5.7-11.4 IU per kg body weight of the patient, per day, may be employed. Most preferably, about 6-10 IU per kg body weight of the patient, per day, may be employed.

When vitamin A is employed, an amount of about 170-357.1 IU per kg body weight of the patient, per day, is employed. More preferably, an amount of about 214.3-357.1 IU per kg body weight of the patient, per day, is employed. Most preferably, an amount of about 220-340 IU per kg body weight of the patient, per day, is employed.

The antioxidants used in the composition of the present invention are preferably selected not only for their antioxidant activity, but also based on other beneficial effects that particular compounds may provide. For example, ascorbyl palmitate not only has antioxidant activity, but also may act as an aldose reductase inhibitor and may help prevent degradation of nitric oxide (NO) and thus is a particularly preferred antioxidant for the present invention. Similarly, vitamin E may also help prevent degradation of nitric oxide and is thus a preferred antioxidant. Vitamin A is also preferred for use as an antioxidant. However, due to its solubility characteristics, vitamin A may need to be formulated in a suitable dispersant such as corn oil in much the same manner as vitamin D₃ as described above.

Suitable additional beneficial properties for compounds useful in the compositions of the present invention include solubility or dispersability, low toxicity, bioavailability when administered, aldose reductase inhibition, antioxidant properties, free radical scavenging, transition metal chelation, nitric oxide stabilization, and anti-inflammatory activity.

The compositions in accordance with the present invention may provide one or more of the following beneficial effects to a patient when administered in effective amounts: relief of pain, burning, tingling, electrical sensations and/or hyperalgesia, increased microcirculation, nitric oxide stabilization, promotion of healing of skin ulcers and lesions, protein kinase C inhibition, decreased oxidative stress, anti-inflammation, blockage of the formation of leukotrienes, stabilization of cell membranes, and promotion of the synthesis of nerve growth factor.

The method of the present invention involves the administration of a composition of the present invention to a patient that suffers from diabetic neuropathy. In the method, a suitable amount of the composition of the invention is administered one to six times daily as needed to relieve pain and other symptoms of the diabetic neuropathy. Preferably, the composition is ingested two to four times daily, as needed for pain. A sufficient amount should be ingested to provide one or more of the beneficial effects of the compositions described above. The method initially treats acute symptoms but may be continued indefinitely to relieve pain, prevent symptoms of diabetic neuropathy from returning and possibly restore some nerve and/or skin function. The method of the present invention may provide one or more of the beneficial effects described above for the compositions of the invention.

The compositions of the present invention may be formulated using the active agents and one or more of the optional ingredients described below or, more preferably, are formulated with a pharmaceutically acceptable carrier. The amount of the active agents that may be combined with the carrier materials to produce a particular dosage form, as well as the treatment regiment, will vary depending on such factors as the patient being treated, the particular mode of administration, the activity of the particular active agents employed, the age, bodyweight, general health, sex, diet, time of administration, rate of excretion, the combination of active agents and the severity of the illness.

The compositions of the present invention may be administered orally, parenterally, by inhalation or via an implanted reservoir. The term “parenteral” as used herein includes subcutaneous, intravenous, intramuscular, intra-articular, intra-synovial, intrrasternal, intrathecal, intrahepatic, intralesional and intracranial injection or infusion techniques. Preferably, the compositions are administered orally, intraperitoneally or intraveneously.

Sterile injectable forms of the compositions of the invention may be in the form of an aqueous or oleaginous suspension. These suspensions may be formulated according to techniques known in the art using suitable dispersing or wetting agents and/or suspending agents, if needed. The sterile injectable preparation may also be a sterile injectable solution or suspension in a non-toxic parenterally acceptable diluent or solvent. Amount the acceptable vehicles or solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides, fatty acids such as oleic acid and its glyceride derivatives are useful in the preparation of injectables, as are natural pharmaceutically acceptable oils such as olive oil or castor oil, especially when polyethoxylated. These oil solutions or suspensions may also contain a long-chain alcohol diluent or dispersant. Also suitable for parenteral administration are non-sterile solutions of the active agents in sesame or peanut oil or in aqueous propylene glycol or N,N-dimethylformamide. Aqueous solutions may include a suitable buffering agent and are preferably rendered isotonic via use of saline or glucose.

The compositions of the present invention may also be orally administered in any orally acceptable dosage form including, but not limited to, capsules, tablets, lozenges, troches, hard candies, powders, sprays, elixirs, syrups, and suspensions or solutions.

In the case of tablets for oral use, common pharmaceutically acceptable carriers include lactose and corn starch. Lubricating agents may also be added to the tablets, including, for example, magnesium stearate, sodium lauryl sulfate and talc. Tablets may also contain excipients such as sodium citrate, calcium carbonate and calcium phosphate. Disintegrants such as starch, alginic acid and complex silicates, may also be employed. Tablets may also include binding agents such as polyvinylpyrrolidone, gelatin and gum acacia.

When the composition of the invention is administered in capsule form, it may be used with or without diluents. For capsules, useful diluents include lactose and dried corn starch. When suspensions are employed, emulsifying and/or suspending agents may be employed in the suspensions. In addition, solid compositions including one or more of the ingredients of the tablets described above may be employed in soft and hard gelatin capsules.

The compositions of the present invention may also be administered by nasal aerosol or inhalation. Such compositions may be prepared using well known techniques. For this method of administration, suitable carriers include saline with one or more preservatives, absorption promoters to enhance bioavailability, fluorocarbons and/or other convention solubilizing or dispersion agents.

In general, the active agents will make up from about 0.5-90% by weight of the total composition to provide the desired unit dosage. The body weight dosages given above are based on a 154 pound patient, which is the accepted standard patient for the purpose of clinical trials. Dosages may be administered 1-10 times per day, more preferably 2-8 times per day and most preferably, 4-8 times per day. The appropriate unit dosage may be determined by dividing the daily dosage by the number of unit doses per day, which will be employed in the particular treatment regimen for a specific patient. Thus, the composition of the invention may comprise anywhere from one tenth of the daily minimum dosage of the various active ingredients up to a maximum of the daily maximum dosage of the various active ingredients.

Other materials, which may optionally be included in the compositions of the present invention include inositol, other B-complex vitamins, and anti-inflammatories. Also, ingredients such as sweeteners, flavorants, coloring agents, dyes, and diluents such as water, ethanol, propylene glycol, glycerin and various combinations thereof may be included in the compositions of the present invention.

The foregoing detailed description of the invention and examples are not intended to limit the scope of the invention in any way and should not be construed as limiting the scope of the invention. The scope of the invention is to be determined from the claims appended hereto.

What is claimed is:

1. A composition for the treatment of diabetic neuropathy by a method of administration selected from the group consisting of oral administration, parenteral administration and inhalation, the compositions comprising a mixture of an amount of a compound that promotes synthesis of nerve growth factor which is effective when administration in the composition to promote synthesis of nerve growth factor, an amount of an aldose reductase inhibitor which is effective when administered in the composition to inhibit aldose reductase and an effective amount of an antioxidant.

2. A composition as claimed in claim 1, wherein the compound that promotes the synthesis of nerve growth factor is selected from the group consisting of: vitamin D₃, 1(S), 3(R)-dihydroxy-20R-(1-ethoxy-5-ethyl-5-hydroxy-2-heptyn-1-yl)-9, 10-seco-pregna-5(Z), 7(E), 10(19)-triene, and other vitamin D₃ derivatives which promote the synthesis of nerve growth factor, pharmaceutically acceptable salts thereof and mixtures thereof.

3. A composition as claimed in claim 1, wherein the aldose reductase inhibitor is selected from the group consisting of: flavonoids, flavonoid derivatives which exhibit aldose reductase inhibiting properties, pharmaceutically acceptable salts thereof and mixtures thereof.

4. A composition as claimed in claim 3, wherein the aldose reductase inhibitor is selected from the group consisting of: (−)-epigallocatechin; (−)-epigallocatechin-gallate; 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8-glucoside; haematoxylin; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; wogonin; pharmaceutically acceptable salts thereof, and mixtures thereof.

5. A composition as claimed in claim 3, wherein the aldose reductase inhibitor comprises at least one compound selected from the group consisting of: quercetin, quercetrin, myricetin, kaempferol and myrecetrin.

6. A composition as claimed in claim 1, wherein the antioxidant comprises at least one compound selected from the group consisting of: ascorbyl palmitate, ascorbic acid (vitamin C), vitamin A, vitamin E acetate, α-lipoic acid, especially DL-α-lipoic acid, coenzyme Q10, glutathione, catechin, glangin, rutin, luteolin, morin, fisetin silymerin, apigenin, gingkolides, hesperitin, cyanidin, citrin, derivatives thereof which exhibit antioxidant activity, and pharmaceutically acceptable salts thereof.

7. A composition as claimed in claim 6, wherein the antioxidant comprises a mixture of at least two different compounds.

8. A composition as claimed in claim 1, wherein the antioxidant comprises vitamin E.

9. A composition as claimed in claim 1, wherein the antioxidant comprises vitamin A.

10. A composition as claimed in claim 1, wherein the antioxidant comprises ascorbyl palmitate.

11. A composition as claimed in claim 8, wherein the antioxidant further comprises at least one compound selected from the group consisting of ascorbyl palmitate and vitamin A.

12. A composition as claimed in claim 5, wherein the compound that promotes the synthesis of nerve growth factor comprises vitamin D₃.

13. A composition as claimed in claim 12, wherein the antioxidant comprises at least one compound selected from the group consisting of vitamin A, vitamin E, and ascorbyl palmitate.

14. A composition as claimed in claim 13, wherein the antioxidant comprises vitamin A, vitamin E as mixed tocopherols and ascorbyl palmitate and the aldose reductase inhibitor comprises quercetin.

15. A composition as claimed in claim 1, further comprising an effective amount of a pharmaceutically acceptable carrier.

16. A method for the treatment of diabetic neuropathy comprises the step of administering by a method of administration selected from the group consisting of oral administration, parenteral administration and inhalation, an effective amount of a mixture which comprises an amount of a compound that promotes synthesis of nerve growth factor which is effective when administered in the composition to promote synthesis of nerve growth factor, an amount of an aldose reductase inhibitor which is effective when administered in the composition to inhibit aldose reductase and an effective amount of an antioxidant.

17. A method as claimed in claim 16, wherein the compound that promotes the synthesis of nerve growth factor is selected from the group consisting of: vitamin D₃, 1(S), 3(R)-dihydroxy-20(R)-(1-ethoxy-5-ethyl-5-hydroxy-2-heptyn-1-yl)-9, 10-seco-pregna-5(Z), 7(E), 10(19)-triene, and other vitamin D₃ derivatives which promote the synthesis of nerve growth factor, pharmaceutically acceptable salts thereof and mixtures thereof.

18. A method as claimed in claim 17, wherein the aldose reductase inhibitor is selected from the group consisting of: flavonoids, flavonoid derivatives which exhibit aldose reductase inhibiting properties, pharmaceutically acceptable salts thereof and mixtures thereof.

19. A method as claimed in claim 18, wherein the aldose reductase inhibitor is selected from the group consisting of: (−)-epigallocatechin; (−)-epigallocatechin-gallate; 1,2,3,6-tetra-o-gallyol-β-d-glucose; 2′o-acetylacetoside; 3,3′,4-tri-o-methyl-ellagic acid; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin-8glucoside; haematoxylin; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; wogonin; pharmaceutically acceptable salts thereof, and mixtures thereof.

20. A method as claimed in claim 16, wherein the antioxidant comprises at least one compound selected from the group consisting of: ascorbyl palmitate, ascorbic acid (vitamin C), vitamin A, vitamin E acetate, α-lipoic acid, especially DL- α-lipoic acid, coenzyme Q10, glutathione, catechin, glangin, rutin, luteolin, morin, fisetin, silymerin, apigenin, gingkolides, hesperitin, cyanidin, citrin, derivatives thereof which exhibit antioxidant activity, and pharmaceutically acceptable salts thereof.

21. A method as claimed in claim 20, wherein the compound that promotes the synthesis of nerve growth factor comprises vitamin D₃, the aldose reductase inhibitor comprises at least one compound selected from the group consisting of: quercetin, quercetrin, myricetin, kaempferol and myrecetrin, and the antioxidant comprises at least one compound selected from the group consisting of vitamin A, vitamin E acetate, and ascorbyl palmitate.

22. A method as claimed in claim 21, wherein the antioxidant comprises vitamin A, vitamin E acetate and ascorbyl palmitate and the aldose reductase inhibitor is quercetin.

23. A method as claimed in claim 16, wherein the composition further comprises an effective amount of a pharmaceutically acceptable carrier.

24. A method as claimed in claim 16, wherein an amount of about 11-28.6 mg/kg body weight of the patient per day of ascorbyl palmitate is employed, an amount of about 170-357.1 IU per kg body weight of the patient, per day, of vitamin A is employed, an amount of about 4-11.4 IU per kg body weight of the patient, per day, of vitamin E is employed, about 13-21.4 mg/kg body weight of the patient per day of quercetin is employed, and about 6-14.3 IU per kg body weight of the patient of vitamin D₃ is employed.

25. A method as claimed in claim 16, wherein an amount of about 14.3-28.6 mg/kg body weight of the patient per day of ascorbyl palmitate is employed, an amount of about 214.3-357.1 IU per kg body weight of the patient, per day, of vitamin A is employed, an amount of about 5.7-11.4 IU per kg body weight of the patient, per day, of vitamin E is employed, about 17.2-21.4 mg/kg body weight of the patient per day of quercetin is employed, and about 8-14.3 IU per kg body weight of the patient of vitamin D₃ is employed.
